(19) United States
(12) Patent Application Publication
(10) Pub. No.: US 2021/0027866 A1
(43) Pub. Date: Jan. 28, 2021

(54) TRANSPORTATION APPARATUS, INFORMATION PROCESSING SYSTEM, STORAGE MEDIUM, AND INFORMATION PROCESSING METHOD

(71) Applicant: TOYOTA JIDOSHA KABUSHIKI KAISHA, Toyota-shi (JP)

(72) Inventors: Yusuke HARA, Musashino-shi (JP); Shuhei AKETA, Koto-ku (JP); Toru YANAGIDA, Nagoya-shi (JP); Shin SAKURADA, Toyota-shi (JP); Tae SUGIMURA, Miyoshi-shi (JP); Yasutaka UJIHARA, Meguro-ku (JP)

(73) Assignee: TOYOTA JIDOSHA KABUSHIKI KAISHA, Toyota-shi (JP)

(21) Appl. No.: 16/935,868

(22) Filed: Jul. 22, 2020

(30) Foreign Application Priority Data
Jul. 22, 2019 (JP) 2019-134662

(51) Int. Cl. G16H 10/20; H04W 4/90; G16H 10/60; G16H 80/00

(52) U.S. Cl. CPC G16H 10/20; G16H 80/00; G16H 10/60; H04W 4/90

(57) ABSTRACT

A transportation apparatus includes an input interface, an output interface, and a controller. The input interface is capable of detecting a user input in a passenger space. The output interface is capable of outputting information in the passenger space. The controller provides a medical interview question via the output interface. The controller provides an advice message in accordance with an answer to the medical interview question, the answer being received as the user input detected by the input interface.

### CROSS-REFERENCE TO RELATED APPLICATION

The present application claims priority to Japanese Patent Application No. 2019-134662 filed on Jul. 22, 2019, the entire disclosure of which is incorporated herein by reference.

### TECHNICAL FIELD

The present disclosure relates to a transportation apparatus, an information processing system, a storage medium, and an information processing method.

### BACKGROUND

A technology for suggesting an appropriate clinical department to a patient who desires to receive a medical service at a medical facility has been developed (refer to PTL 1). Another technology for determining whether emergency transportation is needed for an ill or injured person has also been developed (refer to PTL 2).

### CITATION LIST

### Patent Literature

PTL 1: JP6152678B2

PTL 2: WO2017154711A1

### SUMMARY

The technologies described in PTL 1 and PTL 2 support patients or ill or injured persons who are determined to receive medical services at medical facilities, but do not support people who have no intention to visit medical facilities. People other than medical service providers usually have only shallow medical knowledge, and thus have difficulty in deciding what to do when they feel slightly but not seriously ill. Information indicating the matters to be considered based on the specific health conditions is indeed available on the Internet or other sources; however, it is too bothering to acquire different kinds of information for different health conditions on a daily basis. As a result, it has been difficult to encourage people to have a health checkup unless the health condition is serious enough to cause some trouble in their daily life.

In consideration of the above background, an object of the present disclosure is to encourage people to have a health checkup.

A transportation apparatus according to an embodiment of the present disclosure includes an input interface capable of detecting a user input in a passenger space, an output interface capable of outputting information in the passenger space, and a controller configured to provide a medical interview question via the output interface and provide an advice message based on an answer to the medical interview question, the answer being received as the user input detected by the input interface.

An information processing system according to an embodiment of the present disclosure includes a transportation apparatus and a terminal apparatus. The transportation apparatus includes an input interface capable of detecting a user input in a passenger space, an output interface capable of outputting information in the passenger space, and a controller configured to provide a medical interview question via the output interface and provide an advice message based on an answer to the medical interview question, the answer being received as the user input detected by the input interface. The terminal apparatus communicates with the transportation apparatus.

A non-transitory computer-readable storage medium according to an embodiment of the present disclosure stores a program. The program, when executed, causes a controller installed in a transportation apparatus to execute a process including providing a medical interview question via an output interface capable of outputting information in a passenger space and providing an advice message based on an answer to the medical interview question, the answer being received as a user input detected by an input interface capable of detecting a user input in the passenger space.

An information processing method according to an embodiment of the present disclosure is implemented by a controller installed in a transportation apparatus. The information processing method includes providing a medical interview question via an output interface capable of outputting information in a passenger space and providing an advice message based on an answer to the medical interview question, the answer being received via an input interface capable of detecting a user input in the passenger space.

The transportation apparatus, the information processing system, the storage medium, and the information processing method according to an embodiment of the present disclosure can encourage people to have a health checkup.

### BRIEF DESCRIPTION OF THE DRAWINGS

In the accompanying drawings:

FIG. 1 is a configuration diagram illustrating an overall configuration of an information processing system including a transportation apparatus according to an embodiment of the present disclosure;

FIG. 2 is a functional block diagram schematically illustrating a configuration of the transportation apparatus in FIG. 1;

FIG. 3 is a functional block diagram schematically illustrating a configuration of a first information processing apparatus in FIG. 1;

FIG. 4 is a functional block diagram schematically illustrating a configuration of a terminal apparatus in FIG. 1; and

FIG. 5 is a flowchart illustrating advice processing performed by a controller in FIG. 2.

### DETAILED DESCRIPTION

Hereinafter, an embodiment of the present disclosure will be described with reference to the drawings.

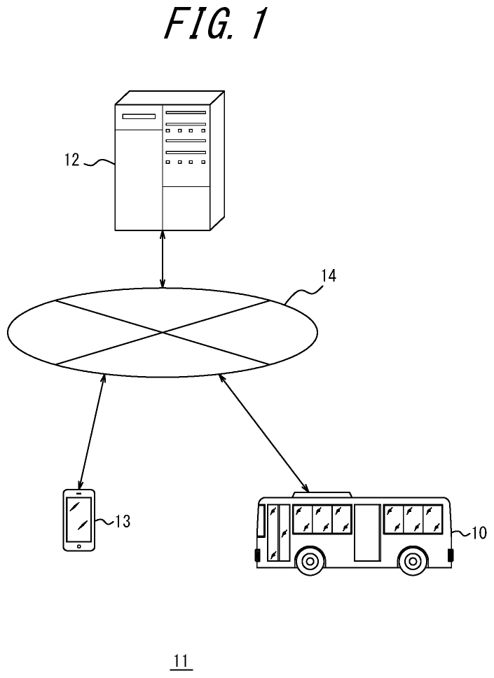

An information processing system 11 including a transportation apparatus 10 according to an embodiment of the present disclosure will be outlined with reference to FIG. 1. The information processing system 11 includes the transportation apparatus 10, a first information processing apparatus 12, and a terminal apparatus 13.

The transportation apparatus 10 is any means of transportation that can transport passengers other than an operator. The transportation apparatus 10 is, for example, a bus, a taxi, a train, a ship, or an airplane. The transportation apparatus 10 may be any means of public transportation or any means of transportation that travels around a specific area to provide health advice. The first information processing apparatus 12 includes one server apparatus or a plurality of server apparatuses that are capable of communicating with each other. The terminal apparatus 13 is, for example, a general electronic device such as a smartphone or a personal computer (PC), but the terminal apparatus 13 is not limited to this example and may be an electronic device dedicated to the information processing system 11. While FIG. 1 illustrates one transportation apparatus 10 and one terminal apparatus 13 for ease of description, the information processing system 11 only has to include at least one transportation apparatus 10 and at least one terminal apparatus 13. The transportation apparatus 10, the first information processing apparatus 12, and the terminal apparatus 13 are communicably connected to a network 14 which includes, for example, a mobile communication network and the Internet.

The outline of the present embodiment is further explained. The transportation apparatus 10 conducts a medical interview with a passenger in a passenger space. The passenger space in the transportation apparatus 10 is a seat or a room where a person other than an operator is present. The transportation apparatus 10 provides the passenger with an advice message based on answers to the medical interview. The transportation apparatus 10 may generate the content of the advice message in the first information processing apparatus 12. It should be noted that, as used herein, “generate” denotes generating information on a target such as the content of an advice message. In a configuration in which the transportation apparatus 10 generates the content of the advice message in the first information processing apparatus 12, a code may be provided as an advice message, to give the terminal apparatus 13 access to the first information processing apparatus 12 via the code. The code may be, for example, a quick response (QR) Code® (QR code is a registered trademark in Japan, other countries, or both). In such a manner, the present embodiment allows a passenger of the transportation apparatus 10 to have a medical interview during travel by the transportation apparatus 10.

Next, the constituents of the information processing system 11 are described in detail.

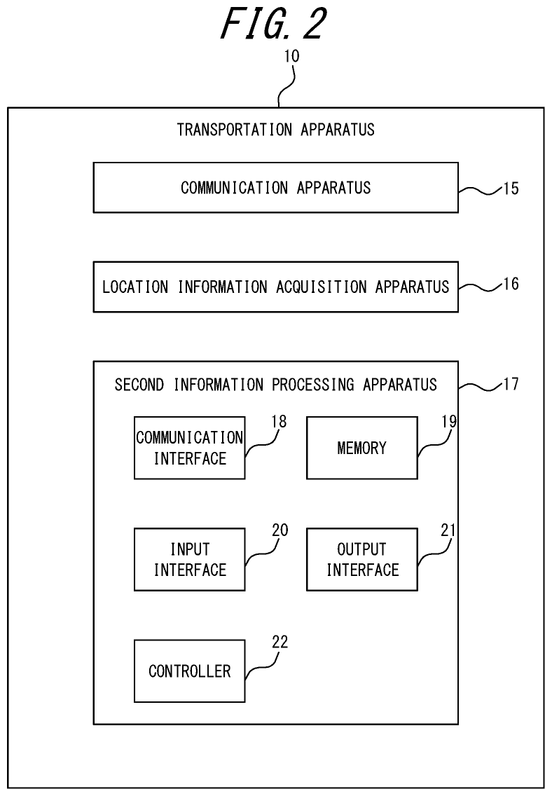

As illustrated in FIG. 2, the transportation apparatus 10 is equipped with a communication apparatus 15, a location information acquisition apparatus 16, and a second information processing apparatus 17. The communication apparatus 15, the location information acquisition apparatus 16, and the second information processing apparatus 17 are communicably connected to one another via, for example, an in-vehicle network such as a controller area network (CAN) or dedicated lines. The communication apparatus 15 is a dedicated on-board communication device.

The communication apparatus 15 includes a communication module that establishes communication via an in-vehicle network or a dedicated line. The communication apparatus 15 also includes a communication module that establishes connection with the network 14. For example, the communication apparatus 15 may include a communication module compliant with mobile communication standards such as the fourth generation (4G) and the fifth generation (5G). In the present embodiment, the transportation apparatus 10 is connected to the network 14 via the communication apparatus 15. When the communication apparatus 15 sends information through the network 14, the communication apparatus 15 may add identification information for the transportation apparatus 10 to the information. The identification information for the transportation apparatus 10 is information that can be used for uniquely identifying the transportation apparatus 10 in the information processing system 11.

The location information acquisition apparatus 16 includes at least one receiver for a satellite navigation system. For example, the location information acquisition apparatus 16 may include a global positioning system (GPS) receiver. The location information acquisition apparatus 16 acquires a measured value corresponding to the location of the transportation apparatus 10 as location information and sends the measured value to the second information processing apparatus 17.

The second information processing apparatus 17 includes a communication interface 18, a memory 19, an input interface 20, an output interface 21, and a controller 22.

The communication interface 18 includes a communication module that establishes communication via an in-vehicle network or a dedicated line.

The memory 19 is, but not limited to, a semiconductor memory, a magnetic memory, an optical memory, or the like. The memory 19 may function as, for example, a primary storage, an auxiliary storage, or a cache memory. The memory 19 may store, for example, a system program and an application program. The information stored in the memory 19 may be updated using, for example, information acquired from the network 14 via the communication apparatus 15.

The input interface 20 includes at least one interface that detects user inputs. For example, the input interface 20 may be, but is not limited to, physical keys, capacitive keys, a touch screen provided in combination with a display of the output interface 21, or a microphone that accepts sound input. The input interface 20 is positioned close to the passenger space of the transportation apparatus 10. Thus, the input interface 20 can receive user inputs in the passenger space.

The output interface 21 includes at least one interface that outputs information to be notified to users. For example, the output interface 21 may be, but is not limited to, a display that outputs information as images or a speaker that outputs information as sound. The output interface 21 is positioned close to the passenger space of the transportation apparatus 10. Thus, the output interface 21 can output information in the passenger space.

The controller 22 includes at least one processor. In the present embodiment, a “processor” may be, but is not limited to, a general processor or a processor dedicated to a particular processing operation. The controller 22 controls the entire operation of the second information processing apparatus 17.

When the input interface 20 detects a user input which requests a medical interview, the controller 22 conducts a medical interview with a user. More specifically, the controller 22 conducts a medical interview by notifying medical interview questions. The medical interview questions are output by the output interface 21 so as to be notified. As used herein, “output” denotes outputting information on a target such as a question.

The medical interview questions include questions regarding, for example, age, height, weight, and sex. The medical interview questions include questions in stages regarding, for example, body parts such as the entire body, the head, and the abdomen, various conditions of the body parts such as aches, the severity level of the condition, and the time of occurrence of the condition. The various conditions corresponding to the body parts include, for example: in the case of the head, abnormalities such as aches and nausea that are considered to occur when something is wrong with the head; in the case of eyes, abnormalities such as pains, weariness, and bleariness that are considered to occur when something is wrong with eyes.

In the case in which a passenger can decide a destination for the transportation apparatus 10, the controller 22 may allow the output interface 21 to output a request for an input of the destination before outputting the medical interview questions. For example, in the case in which the transportation apparatus 10 is a bus, the destination may be any bus stop that comes after the current location. For example, in the case in which the transportation apparatus 10 is a taxi, the destination may be the address of a point of interest or a point on the map. For example, in the case in which the transportation apparatus 10 is a train, the destination may be a train station that comes after the current location.

When a user input of a destination is detected, the controller 22 receives from the location information acquisition apparatus 16 a current location of the transportation apparatus 10. As used herein, “receive” denotes receiving information on a target such as a current location. The controller 22 estimates travel time from the current location to the destination by the transportation apparatus 10. The controller 22 may change the amount of medical interview questions and the contents of medical interview questions in accordance with the estimated travel time. For example, as the travel time increases, the controller 22 may increase the amount of questions. For example, when the travel time increases, the controller 22 may increase the number of words or change the contents to lead to answers that are expected to be sensory and require deeper thinking, such as the type of pain. The amount of questions and the contents of questions may be determined such that an expected time to be taken by a general user to understand and answer the questions and understand an advice message as the result of a medical interview do not exceed the travel time.

When the input interface 20 detects a user input of information which identifies a user during a medical interview, the controller 22 searches for a medical history of the user. As used herein, “search for” denotes searching for and discovering information on a target such as a medical history. The information which identifies a user is information that can be used for uniquely identifying a user and may be, for example, the Japanese resident register code or an identification number registered for an individual user in the information processing system 11. The controller 22 searches for the medical history of the user by, for example, reading out the medical history of the user from the memory 19. As used herein, “read out” denotes reading out information on a target such as a medical history. Alternatively, the controller 22 searches for a medical history, for example, such that the controller 22 causes the communication interface 18 to ask the first information processing apparatus 12 for the user's medical history and receives the user's medical history from the first information processing apparatus 12 via the communication interface 18.

When user inputs of answers to the medical interview questions are detected, the controller 22 generates an advice message in accordance with the answers. The controller 22 may generate an advice message, for example, such that, in accordance with a combination of answers to many questions, the controller 22 reads out, from the memory 19, a particular advice message corresponding to the combination. It should be noted that, in such a configuration, the memory 19 stores in a correspondence table advice messages that are predetermined by a doctor or through machine learning and that respectively correspond to different combinations of various answers to many questions. As used herein, “generate” denotes generating information on a target such as an advice message. When a medical history is discovered, the controller 22 generates an advice message also in accordance with the medical history. The controller 22 notifies the generated advice message for a user by causing the output interface 21 to output the generated advice message.

The advice message may include, for example, at least one of a proposal for medical consultation at a medical facility, suggestions in daily life, precautions, and a proposal for medication. The proposal for medical consultation at a medical facility may include, for example, the type of medical facility such as hospital or clinic or a particular clinical department to visit. The suggestions in daily life may include, for example, details of meals and the amount of each meal, and the recommended type and strength of exercise. The precautions may include, for example, a proposal for medical consultation at a medical facility if the current condition lasts for one month or if nausea is caused in addition to the current condition. The proposal for medication is, for example, a suggestion for taking medicines purchasable without prescriptions.

The controller 22 may provide an advice message via the terminal apparatus 13. The controller 22 may send an advice message to the terminal apparatus 13 by, for example, establishing communication such as near-field communication. Alternatively, the controller 22 may, for example, generate a code such as a QR Code® and output the code via the output interface 21. The code is used for causing the terminal apparatus 13 to output an advice message. The code may be, for example, decoded by the terminal apparatus 13 and converted into an image for providing an advice message. Alternatively, the code may be, for example, recognized by the terminal apparatus 13 by means of image capturing to lead to a web page containing an image for providing an advice message. The controller 22 may lead to the web page and display the image. The controller 22 may, for example, generate the web page used for providing an advice message and store the web page in the memory 19. Otherwise, the controller 22 may send the web page to the first information processing apparatus 12.

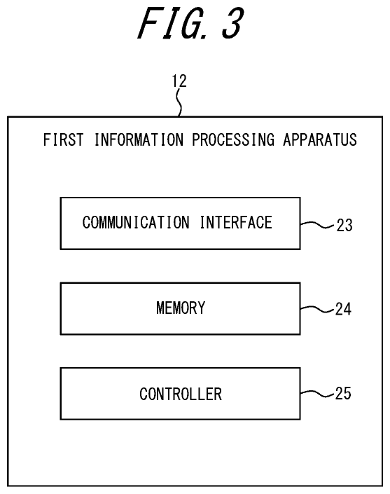

As illustrated in FIG. 3, the first information processing apparatus 12 includes a communication interface 23, a memory 24, and a controller 25.

The communication interface 23 includes a communication module that establishes communication via a dedicated line. The communication interface 23 also includes a communication module that establishes connection with the network 14. For example, the communication interface 23 may include a communication module compliant with mobile communication standards such as 4G and 5G. In the present embodiment, the first information processing apparatus 12 is connected to the network 14 via the communication interface 23.

The memory 24 may be, but is not limited to, a semiconductor memory, a magnetic memory, an optical memory, or the like. The memory 24 may function as, for example, a primary storage, an auxiliary storage, or a cache memory. The memory 24 stores any information that is used for an operation of the first information processing apparatus 12. The memory 24 may store, for example, a system program and an application program.

The controller 25 includes at least one processor. In the present embodiment, a “processor” is, but not limited to, a general processor or a processor dedicated to a particular processing operation. The controller 25 controls the entire operation of the first information processing apparatus 12.

In accordance with a registration request received from a medical facility, the controller 25 registers the medical facility. The controller 25 receives the registration request, for example, from a terminal apparatus installed in a medical facility or by a user input via a keyboard or the like of an administrator of the first information processing apparatus 12. The controller 25 performs registration by storing in the memory 24 identification information for a medical facility included in the registration request. The identification information for a medical facility is information that can be used for uniquely identifying the medical facility in the information processing system 11.

The controller 25 receives a medical history of an individual patient from the registered medical facility. The controller 25 receives a medical history regularly or cyclically. The controller 25 collects in the memory 24 the received medical history in association with the identification information for identifying the patient. Alternatively, the controller 25 may send the received medical history to the second information processing apparatus 17.

When an inquiry about a medical history is received from the second information processing apparatus 17, the controller 25 searches the memory 24 for a medical history of a particular user in accordance with the information for identifying the particular user included in the inquiry. The controller 25 sends the discovered medical history to the second information processing apparatus 17.

When a web page for outputting the content of an advice message is received from the second information processing apparatus 17, the controller 25 stores the web page in the memory 24. When a request for provision of a web page is received from the terminal apparatus 13, the controller 25 searches the memory 24 for a web page and sends the web page to the terminal apparatus 13.

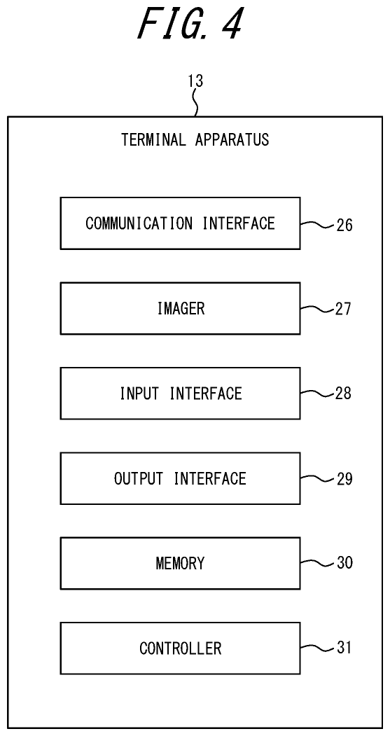

As illustrated in FIG. 4, the terminal apparatus 13 includes a communication interface 26, an imager 27, an input interface 28, an output interface 29, a memory 30, and a controller 31.

The communication interface 26 includes a communication module that establishes communication via a dedicated line. The communication interface 26 also includes a communication module that establishes connection with the network 14. For example, the communication interface 26 may include a communication module compliant with mobile communication standards such as 4G and 5G. In the present embodiment, the terminal apparatus 13 is connected to the network 14 via the communication interface 26.

The imager 27 includes an optical system and an image sensor. The imager 27 generates an image signal by photoelectrically converting an object image formed on a light-receiving surface of the image sensor with the use of the optical system. The imager 27 transmits the generated image signal to the controller 31.

The input interface 28 includes at least one interface that detects user inputs. For example, the input interface 28 may be, but is not limited to, physical keys, capacitive keys, a touch screen provided in combination with a display of the output interface 29, or a microphone that accepts sound input.

The output interface 29 includes at least one interface that outputs information to be notified to users. For example, the output interface 29 may be, but is not limited to, a display that outputs information as images or a speaker that outputs information as sound.

The memory 30 may be, but is not limited to, a semiconductor memory, a magnetic memory, an optical memory, or the like. The memory 30 may function as, for example, a primary storage, an auxiliary storage, or a cache memory. The memory 30 stores any information that is used for an operation of the terminal apparatus 13. The memory 30 may store, for example, a system program and an application program.

The controller 31 includes at least one processor. In the present embodiment, a “processor” may be, but is not limited to, a general processor or a processor dedicated to a particular processing operation. The controller 31 controls the entire operation of the terminal apparatus 13.

When a user input for performing imaging is detected while an application of a camera is running, the controller 31 causes the imager 27 to capture an object image. When an image corresponding to an image signal generated by imaging contains a code, the controller 31 decodes the code. In the case in which the decoded code corresponds to a Uniform Resource Locator (URL), the controller 31 requests the web page from the URL. The controller 31 receives the web page in response to the request, starts a browser application, and causes the output interface 29 to output the web page.

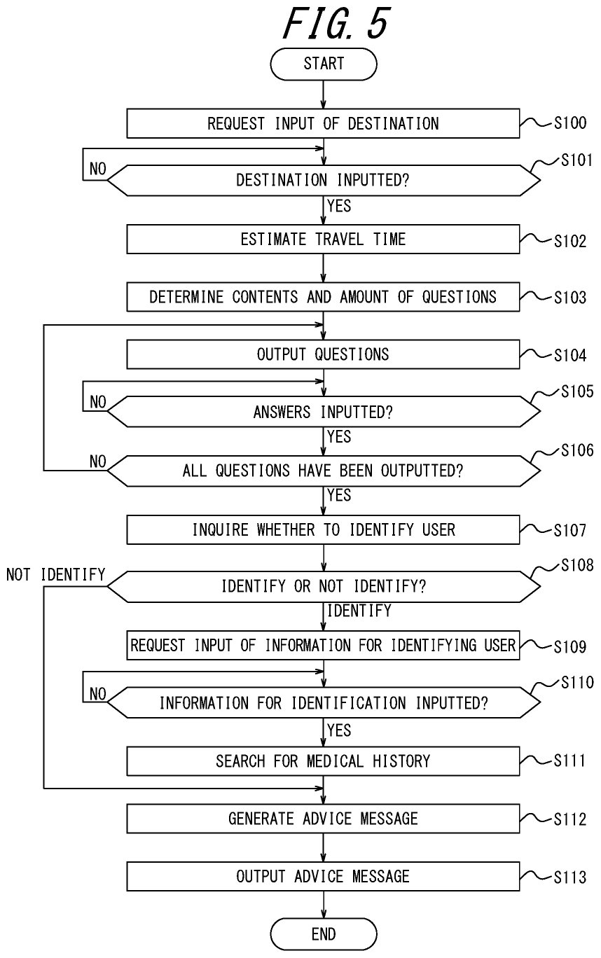

Next, advice processing to be performed by the controller 22 of the second information processing apparatus 17 in the present embodiment is described with reference to the flowchart of FIG. 5. The advice processing is started when, for example, the input interface 20 detects a user input which requests a medical interview.

In step S100, the controller 22 causes the output interface 21 to output a request for an input of a destination. In addition, the controller 22 receives a current location from the location information acquisition apparatus 16. After the request is output, the process proceeds to step S101.

In step S101, the controller 22 determines whether the input interface 20 has detected a user input of a destination. In the case in which the user input has not been detected, the process returns to step S101. In the case in which the user input has been detected, the process proceeds to step S102.

In step S102, the controller 22 estimates travel time in accordance with the current location received in step S100 and the destination indicated by the user input that is determined as having been detected in step S101. After the travel time is estimated, the process proceeds to step S103.

In step S103, the controller 22 determines the contents and the amount of medical interview questions in accordance with the travel time estimated in step S102. After the contents and the amount are determined, the process proceeds to step S104.

In step S104, the controller 22 causes the output interface 21 to output the medical interview question. After the medical interview question is output, the process proceeds to step S105.

In step S105, the controller 22 determines whether the input interface 20 has detected user inputs of answers to the question output in step S104. In the case in which the user inputs have not been detected, the process returns to step S105. In the case in which the user inputs have been detected, the process proceeds to step S106.

In step S106, the controller 22 determines whether outputting all the question determined in step S103 has been completed. In the case in which the outputting has not been completed, the process returns to step S104. In the case in which the outputting has been completed, the process proceeds to step S107.

In step S107, the controller 22 causes the output interface 21 to output an inquiry of whether to identify a user. After the inquiry is output, the process proceeds to step S108.

In step S108, the controller 22 determines whether the detected user input indicates identifying the user or not identifying the user. In the case in which the detected user input indicates identifying the user, the process proceeds to step S109. In the case in which the detected user input indicates not identifying the user, the process proceeds to step S112.

In step S109, the controller 22 causes the output interface 21 to output a request for an input of information for identifying the user. After the request is output, the process proceeds to step S110.

In step S110, the controller 22 determines whether the input interface 20 has detected a user input of the information for identifying the user. In the case in which the user input has not been detected, the process returns to step S110. In the case in which the user input has been detected, the process proceeds to step S111.

In step S111, the controller 22 searches for a medical history of the user in accordance with the information for identifying the user of the user input determined in step S110 to have been detected. After the medical history is retrieved, the process proceeds to step S112.

In step S112, the controller 22 generates an advice message for the user in accordance with the answers of the user inputs determined in step S105. It should be noted that, in the case in which a medical history is retrieved in step S111, the controller 22 generates an advice message for the user also in accordance with the medical history. After the advice message is generated, the process proceeds to step S113.

In step S113, the controller 22 causes the output interface 21 to output the advice message generated in step S112. After the advice message is output, the advice processing is ended.

The transportation apparatus 10 according to the present embodiment configured as described above includes the input interface 20 capable of detecting a user input in a passenger space and the output interface 21 capable of outputting information in the passenger space. The transportation apparatus 10 provides question via the output interface 21 and provides an advice message based on answers to the question received via the input interface 20. During the travel time, a passenger generally does not need to do any specific activity or task. Thus, the passenger is more likely to desire to do something to pass the time during the travel time in the transportation apparatus 10, than in normal times. As a result, the transportation apparatus 10 configured as described above can encourage a user to have a health checkup, who otherwise would not be willing to have a health checkup in normal times.

Furthermore, in the transportation apparatus 10 of the present embodiment, the advice message is a code used for displaying at the terminal apparatus 13 images for providing the content. With this configuration, by using the transportation apparatus 10, the user can check the content of the advice message via the terminal apparatus 13, and thus, the user can check the content of the advice message even after the user gets off the transportation apparatus 10.

Moreover, in the transportation apparatus 10 of the present embodiment, when information for identifying a user is recognized, the controller 22 provides an advice message based also on the medical history of the user. With this configuration, the transportation apparatus 10 can provide an advice message based on a health condition more suitable for a particular user, rather than based on general human health condition simply derived from answers in the medical interview.

Further, the transportation apparatus 10 of the present embodiment estimates travel time to a recognized destination by the transportation apparatus 10 and changes at least one of the content and the amount of questions in accordance with the travel time. In the case in which a passenger of the transportation apparatus 10 had a medical interview conducted by the transportation apparatus 10 but the medical interview was not completed within the travel time, answers to the questions would go wasted. As a result, even if the passenger has an opportunity to take the transportation apparatus 10 at some other time, the experience described above may discourage the passenger to have a health checkup. According to the transportation apparatus 10 of this embodiment, all questions are answered and an advice message is provided within the travel time. As a result, the passenger is less likely to be discouraged to have a health checkup.

While the present disclosure has been described with reference to the accompanying drawings and the examples, it should be understood that various changes and modifications based on the present disclosure may be easily made by those skilled in the art. It should be noted that these changes and modifications are therefore embraced in the scope of the present disclosure. For example, the functions and the like included in the constituents, steps, and the like may be rearranged in a logically consistent manner; a plurality of constituents, steps, or the like may be combined together or divided.

For example, the controller 22, which generates an advice message in accordance with answers to questions or the answers and the medical history, may generate an advice message in accordance with other information relating to the user. For example, the transportation apparatus 10 may include measuring apparatuses for testing blood, measuring pulse, testing urine, testing breath, and the like, and the controller 22 may generate an advice message in accordance with results of such measurements and tests received from the measuring apparatus.

Moreover, some of the processing operations performed by the second information processing apparatus 17 in the embodiment described above may be carried out by the first information processing apparatus 12. Specifically, the first information processing apparatus 12 may provide medical interview questions for the user via the output interface 21 of the second information processing apparatus 17; the first information processing apparatus 12 may receive answers to the questions received via the input interface 20 of the second information processing apparatus 17; and the first information processing apparatus 12 may generate an advice message and provides the advice message for the user via the second information processing apparatus 17.

Furthermore, for example, a general electronic device such as a smartphone or a computer may be configured to function as the first information processing apparatus 12, the terminal apparatus 13, or the second information processing apparatus 17 according to the embodiment described above. Specifically, a program in which details of processing for implementing the function of, for example, the second information processing apparatus 17 according to the embodiment are written is stored in a memory of an electronic device; a processor of the electronic device reads and executes the program. Thus, the disclosure according to the present embodiment may be implemented as a program that can be executed by a processor. The program may be downloaded via a network 14; or the program may be stored in portable a non-transitory recording/storage medium readable by electronic devices and the program may be read out from the medium by an electronic device.

1. A transportation apparatus comprising:
an input interface capable of detecting a user input in a passenger space;
an output interface capable of outputting information in the passenger space; and
a controller configured to provide a medical interview question via the output interface and provide an advice message based on an answer to the medical interview question, the answer being received as the user input detected by the input interface.

2. The transportation apparatus according to claim 1, wherein
the controller causes a terminal apparatus to output the advice message.

3. The transportation apparatus according to claim 1, wherein
when the controller recognizes information for identifying a user, the controller searches for a medical history of the user and provides the advice message based also on the medical history.

4. The transportation apparatus according to claim 1, wherein
when the controller recognizes a destination of a user, the controller estimates travel time to the destination by the transportation apparatus and changes at least one of a content and an amount of the medical interview question in accordance with the travel time.

5. The transportation apparatus according to claim 1, wherein
the advice message includes at least any of a proposal for medical consultation, suggestions about daily life, precautions, and a proposal for medication.

6. An information processing system comprising:
the transportation apparatus according to claim 1; and
a terminal apparatus configured to communicate with the transportation apparatus.

7. The information processing system according to claim 6, wherein
the controller causes the terminal apparatus to output the advice message.

8. The information processing system according to claim 6, wherein
when the controller recognizes information for identifying a user, the controller searches for a medical history of the user and provides the advice message based also on the medical history.

9. The information processing system according to claim 6, wherein
when the controller recognizes a destination of a user, the controller estimates travel time to the destination by the transportation apparatus and changes at least one of a content and an amount of the medical interview question in accordance with the travel time.

10. The information processing system according to claim 6, wherein
the advice message includes at least any of a proposal for medical consultation, suggestions about daily life, precautions, and proposal for medication.

11. A non-transitory computer-readable storage medium storing a program which, when executed, causes a controller installed in a transportation apparatus to execute a process, the process comprising:
providing a medical interview question via an output interface capable of outputting information in a passenger space; and
providing an advice message based on an answer to the medical interview question, the answer being received as a user input detected by an input interface capable of detecting a user input in the passenger space.

12. The non-transitory computer-readable storage medium according to claim 11, wherein the process further comprises:
causing a terminal apparatus to output the advice message.

13. The non-transitory computer-readable storage medium according to claim 11, wherein the process further comprises:
when information for identifying a user is recognized, searching for a medical history of the user; and
providing the advice message based also on the medical history.

14. The non-transitory computer-readable storage medium according to claim 11, wherein the process further comprises:
when a destination of a user is recognized, estimating travel time to the destination by the transportation apparatus; and
changing at least one of a content and an amount of the medical interview question in accordance with the travel time.

15. The non-transitory computer-readable storage medium according to claim 11, wherein
the advice message includes at least any of a proposal for medical consultation, suggestions about daily life, precautions, and a proposal for medication.

16. An information processing method implemented by a controller installed in a transportation apparatus, the information processing method comprising:
providing a medical interview question via an output interface capable of outputting information in a passenger space; and
providing an advice message based on an answer to the medical interview question, the answer being received as a user input detected by an input interface capable of detecting a user input in the passenger space.

17. The information processing method according to claim 16, further comprising:
causing a terminal apparatus to output the advice message.

18. The information processing method according to claim 16, further comprising:
when information for identifying a user is recognized, searching for a medical history of the user; and
providing the advice message based also on the medical history.

19. The information processing method according to claim 16, further comprising:
when a destination of a user is recognized, estimating travel time to the destination by the transportation apparatus; and
changing at least one of a content and an amount of the medical interview question in accordance with the travel time.

20. The information processing method according to claim 16, wherein
the advice message includes at least any of a proposal for medical consultation, suggestions about daily life, precautions, and a proposal for medication.
